(19) United States
(12) Patent Application Publication
(10) Pub. No.: US 2018/0188125 A1
(43) Pub. Date: Jul. 5, 2018

(54) Flexible and Stretchable Sensor Using Soft Optical Waveguides

(71) Applicant: CARNEGIE MELLON UNIVERSITY, Pittsburgh, PA (US)

(72) Inventors: Yong-Lae Park, Lexington, MA (US); Celeste To, San Gabriel, CA (US)

(21) Appl. No.: 15/580,233

(22) PCT Filed: Jul. 22, 2016
(86) PCT No.: PCT/US16/43570

§ 371 Date: Dec. 6, 2017

Related U.S. Application Data
(60) Provisional application No. 62/282,014, filed on Jul. 22, 2015.

(51) Int. Cl. G01L 1/24; G01L 11/02; G01B 11/24; A61B 10/02; B29D 11/00

(52) U.S. Cl. CPC G01L 1/242; G01L 11/025; G01B 11/24; A61B 2090/3614; B29D 11/00663; B29D 11/00875; A61B 10/0233

(57) ABSTRACT

A stretchable optical sensor that can detect multiple modes of deformation and contact, including pressure, strain, and bending. The method of operation involves a waveguide and a flexible housing, in one embodiment made of silicone rubber. The interface between the two is a reflective layer that encapsulates light propagating through the channel. As the sensor is stretched, compressed, or bent, cracks within the reflective layer form and allow light to escape, resulting in a linear changes to the signal response.

### RELATED APPLICATIONS

This application claims the benefit of U.S. Provisional Patent Application Ser. No. 62/282,014, filed Jul. 22, 2015.

### BACKGROUND OF THE INVENTION

The advent of soft robotics stemmed from the need of robotic systems that closely interact with human beings with increased safety and friendliness. Naturally the materials and methods used in soft robots differ greatly from those used in traditional robots that are typically made of rigid structures and materials. One of the most important elements in soft robotics, and also in robotics in general, is performance of sensors as well as actuator technologies. Soft sensors that have been developed so far have utilized a range of polymer materials, such as Polydimethylsiloxane (PDMS) and silicone rubbers.

However, since most polymers are nonconductive and require any type of conductive media that can transmit electric signals through the material without significantly changing the mechanical the materials' “soft” properties (i.e. flexibility and stretchability). For this reason, conductive liquids have been one of the most preferred and commonly used mediums due to their continuous property. They can be easily transformed to completely soft electrical wires in a highly deformable structure when encapsulated and sealed by a closed channel embedded in a soft structure. The conductive liquid channels can also work as a sensing element by changing their electrical resistance when the host material deforms. Many soft sensors have been developed using liquid metals embedded microchannels, such as a multi-modal sensor for strain and pressure, a multi-axis shear force sensor, a capacitive force sensor, and a curvature sensor. In addition to liquid metals, ionic liquids have been also used for soft microfluidic sensors for increased biocompatibility and high electrical resistance.

In spite of many advantages of liquid conductors, they have several limitations when coupled with soft materials. Encapsulation is one of the major limitations. Liquid metals are usually injected to microchannels in a soft structure using a thin syringe needle. Due to the hydrophobic nature of the soft material used, the injection requires a relatively high pressure. Also, the captured air in the microchannel needs to be removed simultaneously with injection, which makes it difficult to control the injection pressure. This also makes the manufacturing process time-consuming and complicated, requiring complete sealing of injection ports. Another limitation is biocompatibility. Although liquid metals are not considered as highly toxic, they could be dangerous if they come into contact with skin or ingested. Although ionic liquids are safer to use with the human body for both internally and externally, there still exist leaking and sealing problems.

### SUMMARY OF THE INVENTION

This invention is an optical soft sensor for detecting various deformation modes. In its preferred implementation, the sensor is nontoxic and biocompatible. Because there is no liquid embedded in the sensor, the issues that confound microfluidic sensing are addressed. The idea behind this novel sensor is to detect light transmission through a reflective waveguide made in a transparent soft material. Any mechanical perturbation made to the waveguide structure causes a change in the light detected. The novelty in this invention is introduction of hyperelastic materials and a stretchable waveguide in optical sensing, which is simple to manufacture. Another reason of selecting optical transmission as a sensing mechanism is immunity to electromagnetic interference, as discussed in various applications. A curvature sensing mechanism for a flexible biopsy needle has been proposed by detecting the loss of optical power through pre-cut slits of an embedded optical fiber when the needle was bent. However, conventional optical fibers pose a limitation in our application due to their non-stretchability. Therefore, the nascent field of soft robotics shines light on the possibility to replace traditionally stiff materials with highly deformable ones. Other applications that require flexible components will benefit as well.

There have been some efforts to provide stretchability to optical sensors using PDMS, one of the most commonly used soft materials in optics due to its transparency, low absorption loss, and almost negligible birefringence. Pressure-sensitive skin for stretchable electronics has been proposed by making waveguides in a PDMS layer. However, the proposed mechanism cannot be used for large deformations due to the limited stretchability of PDMS.

The soft sensor proposed in this patent application introduces a hyperelastic sensor capable of detecting various modes of deformation: pressure, strain, and curvature. Another achievement of this invention is the simplicity and cleanness in fabrication.

### BRIEF DESCRIPTION OF THE DRAWINGS

FIG. 1 shows one implementation of the optical soft sensor using a flexible and stretchable waveguide in various deformations: (a) is an active sensor, (b) curvature sensing and (c) strain sensing.

FIG. 2 illustrates a longitudinal cross section with a light source on the left and detector on the right. The red arrows indicate light being emitted. As the sensor is stretched (with Force F), cracks form at the reflective gold interface, and allow some light to escape, which changes the signal reading at the detector.

FIG. 3 presents the fabrication process of the device.

FIG. 4 shows the bottom segment of sensor, with the open channel and close-up of the quality of the reflective gold layer.

FIG. 5 shows a first embodiment of the invention.

FIG. 6 shows the three modes of testing, (a) Pressure test set-up; (b) Strain test set-up; (c) Curvature test set-up.

FIG. 7 shows a second embodiment of the invention.

FIG. 8(a) shows clear elastomer channels with embedded electronics that will be used as waveguides.

FIG. 8(b) is a photo of the optical channel and microscopic images of the metal layer showing the waveguide in an unstretched state, and in a stretched state, showing the microcracks which form in the reflective metal layer.

FIG. 9 shows an alternate embodiment of the invention utilizing optical fibers to deliver and sense the light.

FIGS. 10(a) and (b) show a waveguide made using a sputtering method.

FIGS. 11(a)-11(d) further illustrate the sputtered waveguide.

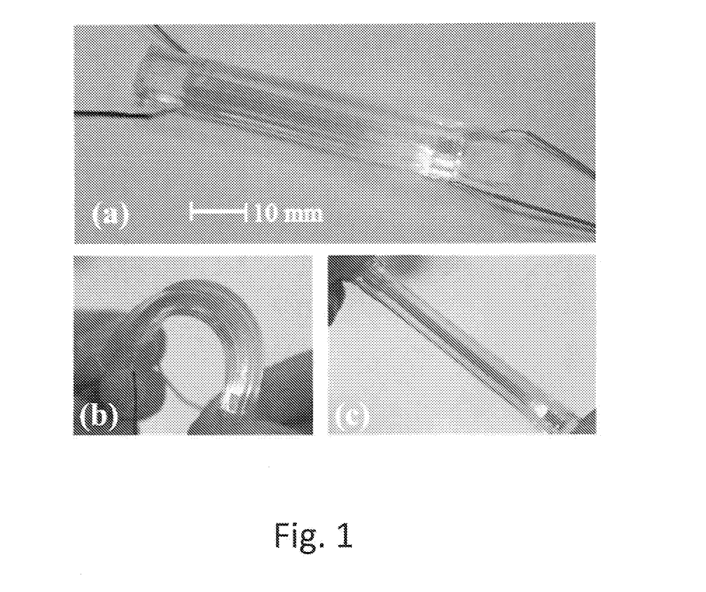

### DETAILED DESCRIPTION OF THE INVENTION

The invention uses the notion of transmitting light from one end of the device through a transparent waveguide and detecting the change in light intensity at the other end of the device as the sensor undergoes deformation in the form of compression, stretching or curvature. The change in light intensity at the other end is due to microcracks which form in a reflective layer covering the waveguide as the device is deformed, allowing light to escape the waveguide and be absorbed by the housing of the device.

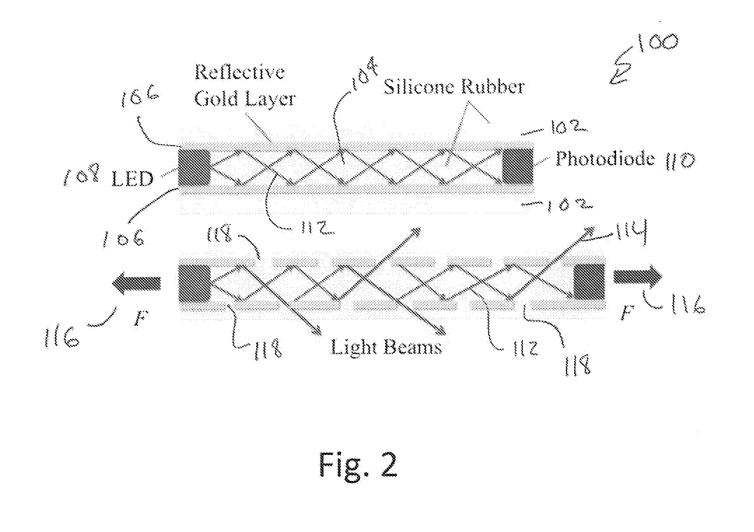

FIG. 2 shows the construction of the device 100. Waveguide 104 and outside housing 102 consist of a stretchable, transparent elastomer material. The outside walls of waveguide 104 are coated with a reflective layer 106, preferably a reflective metal, such as aluminum or silver, and more preferably, gold. However, any highly reflective material could be used. A light source 108, preferably an LED, is located at one end of waveguide 104, and a photodiode 110 that detects the light intensity is positioned at the opposite end of waveguide 104. In its non-active state, the light 112 emitted by light source 108 is reflected internally and detected at the other end without loss due to the encapsulating reflective walls 106.

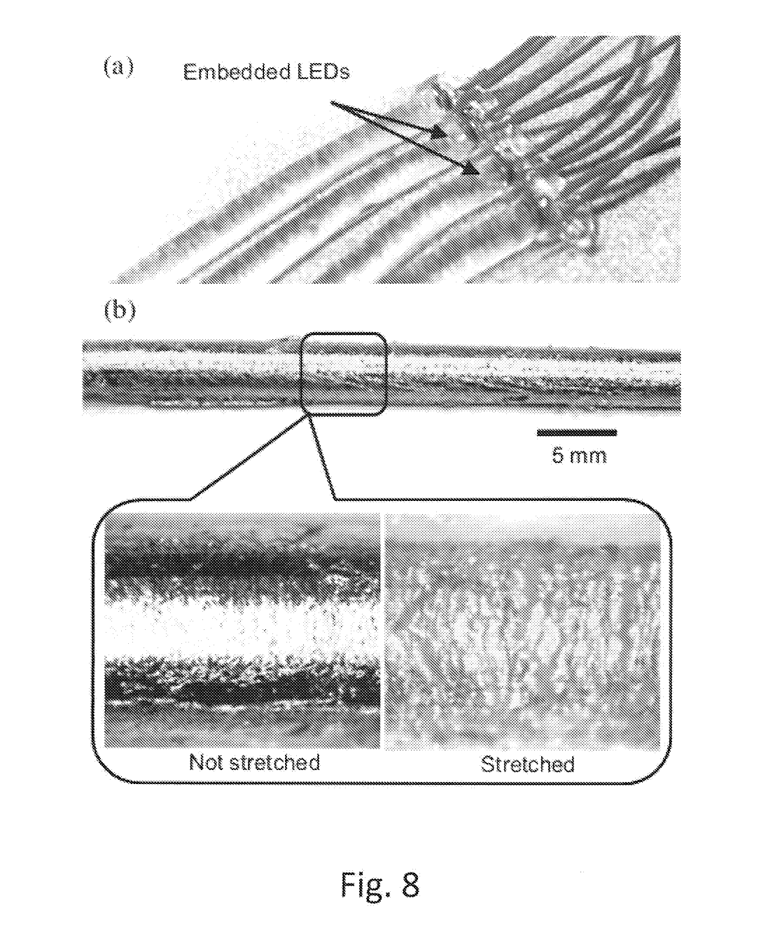

However, as the device 100 is deformed, microcracks 118 will form within reflective layer 106. A microscopic photo of this effect is shown in FIG. 8(b). The microcracks 118 that form along the channel allow the light to escape 114, which results in a drop in light intensity. This drop is a function of the deformation applied to the sensor. Modes of deformation that can be discerned include pressure (i.e., compression), stretching, and curvature (bending). The device is simple, clean, and easy to manufacture.

The method of operation for this invention works by modulating the light intensity at photodiode 110 when the structure of the sensor is deformed by an external force. The measures of those deformations are defined as specifically pressure, strain, and curvature.

In one implementation, the elements used for the optical sensing method are a red surface mount LED 108, for example, an XLamp XBD, manufactured by CREE, and a photodiode 110 sensitive to red light, for example, an CLS15, manufactured by Everlight. Surface mount elements were used in this particular implementation to minimize the form factor, however, any form factor will work. Both the photodiode 110 and red LED 108 have, in the preferred embodiment, dimensions smaller than 3.2 mm. Red LED 108 has a peak wavelength of 625 nm and photodiode 110 has peak sensitivity for 620 nm. In alternative embodiments of the invention light sources of different wavelengths may be used, depending on the application. In such cases, a photodetector capable of detecting the appropriate frequencies will need to be paired with the light source.

Each element is placed at one end of the channel. Photodiode 110 was operated in reverse bias to provide a higher linear response than in forward bias. It was also connected to a current to voltage operational amplifier, for example, an LM358AP. The current generated by photodiode 110 is directly proportional to the optical power provided by LED 108. In an ideal condition the current generated by LED 108 is

$${i}_{s} = \frac{eP}{hv}$$

where e is the charge of an electron, h is the energy of a photon, and P is the optical power with a unit of W/m. Ideally, the signal should be linear. Thus, any nonlinearity in the relationship between the signal output and the optical power input would be due to either absorption loss or optical loss through microcracks 118. A more accurate way to calculate the current is by including the dark current that is determined by the material of the photodiode itself. Electron volts (eV) for example is the valence band for a given element. In this case, the photodiode is made of silicon, which has a valence band of 1.12:

i=iₒ*(e^{eV/kT}−1)−iₛ

Photodiode 110 can be also operated in forward bias (i.e. no-bias) in which a voltage output had a logarithmic relationship with light intensity as:

$$V = \frac{q}{kT} ( ln \frac{i}{{i}_{s}} + 1 )$$

where V, q, k, T, i, and iₛ are voltage output, charge, Boltzman's constant, temperature, current, and saturation current, respectively. We also know that current is linearly proportional to the light intensity. For modeling our system in strain, we can assume that the increase of cracks created within the waveguide is inversely proportional to the light intensity, and the current ratio can be simply replaced by the ratio of the original and stretched surface areas of the waveguide. Assuming the surface area change is dominated by the length change, the ratio further reduces to that of lengths, the original length (lₒ) divided by the stretched length (l). Also, we can replace q/kT with our known initial voltage V₀ for simplification. Finally, the theoretical model can be expressed as:

$$V = {V}_{0} ( ln \frac{{l}_{0}}{l} + 1 )$$

There have been many studies on the optical properties of PDMS which have shown its compatibility with optical elements. PDMS however, lacks high stretchability. In various embodiments, this invention uses different materials to expand on the possible silicone rubbers that are optically compatible as well. The substrate material used in one embodiment of the invention is a hybrid between two silicone rubbers. The first one (Solaris, Smooth On) is optically transparent but not highly stretchable. The other (EcoFlex Gel, Smooth On) is much more stretchable with optical transparency but too soft and tacky. The hybrid was a 1:4 mix of Solaris and EcoFlex Gel. The two combined generated a transparent polymer with a relatively high elongation at break at around 300%. This custom silicone rubber obtained high stretchability while maintaining optical transparency.

While optical fibers achieve total internal reflection by having the refractive index of the waveguide higher than the outer cladding, this invention achieves total internal reflection by using a reflective coating 106 between the housing 102 and the waveguide 104. In one embodiment, 24 k gold leaf was used as reflective coating 106. The gold leaf sheets were 0.12 μm in thickness. Gold was chosen for various reasons, the first being that it is biocompatible and used in different medical instruments. Additionally, gold reflects 95% of wavelengths that are longer than 500 nm (i.e. infrared and visible red light). Finally, gold is ideal because it does not tarnish unlike aluminum and silver, which are alternative reflectors.

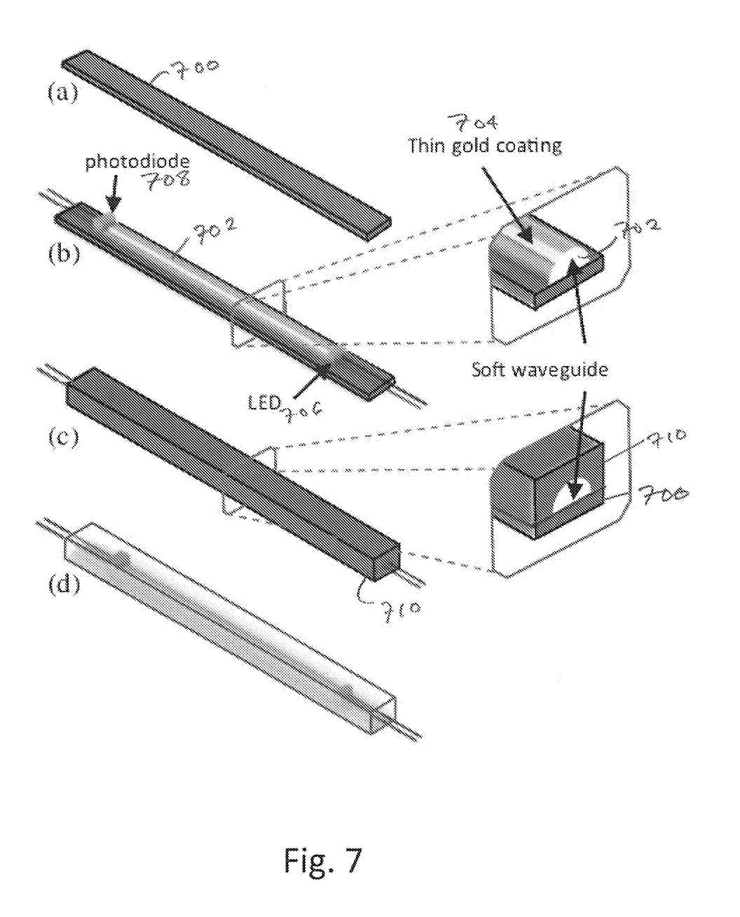

In this embodiment, to create a sensor that can be comparable to those using liquid conductors, the sensor geometry and size was designed to be as small as possible. Surface mount diodes were used because they are significantly smaller than LEDs with pins. Thus, the channel size was accommodated to have a close fit with these circuit elements. The cross sectional area of the channel is square because the diodes are square and would be a closer fit than a circular channel. In other embodiments of the invention, as shown in FIG. 7, the waveguide can have any cross-sectional shape.

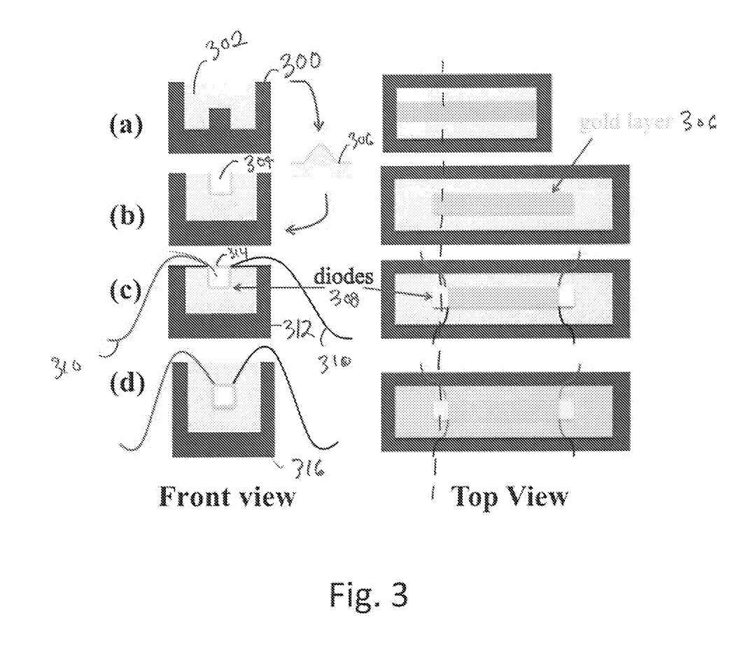

The fabrication process of one embodiment of the invention is shown in FIG. 3. In one embodiment, a 1:4 ratio of Solaris and EcoFlex Gel was used for the substrate. In between each casting, the optical elements and the reflective layer are added.

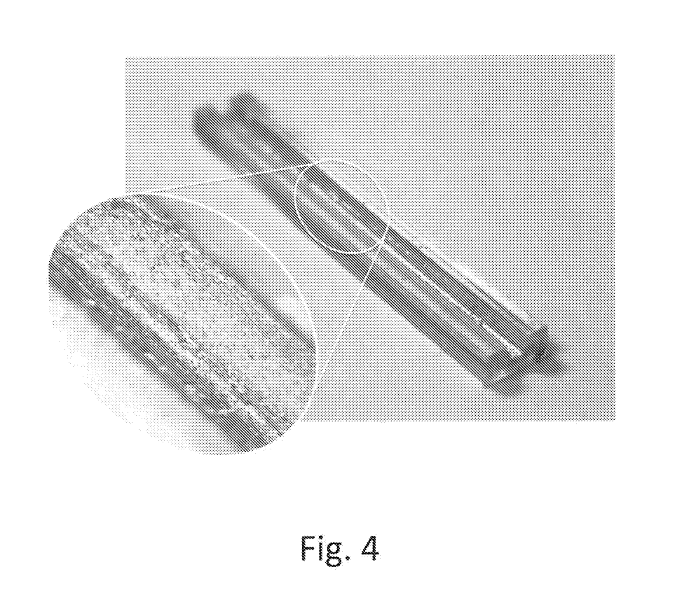

Three separate molds were made. Before curing at each stage, molds were placed in a vacuum to remove bubbles. The first mold 300, shown in FIG. 3(a), casts the bottom of the sensor body 302 so that there is a complete channel upon removal. When removed, the segment was opened up and pinned on the sides to reveal the inside of the channel 304, as shown in FIG. 3(b). The gold leaf 306 was then applied, which acts as the reflective layer 106 as depicted in FIG. 2. FIG. 4 shows the quality of the gold layer applied. The wrinkles are a result of applying the gold when stretched.

In this embodiment, the next step is embedding the optical elements. Surface mount diodes 308, which were utilized for their small form factor, were soldered to standard 30 AWG hook up wires 310. Epoxy was used to protect the unconventional connections between wires and surface mount elements. Before being embedded, LED 108 and photodiode 110 were covered by a thin plastic shield to prevent light from escaping through the gap between the electronics and the edge of gold layer 306. The shields were made from transparency and coated in white to prevent from interfering ambient light. Another benefit for having these shields was to prevent from the wires coming into contact with the gold interface.

Once the diodes were soldered and shielded, they were fixed into the ends of the channel, as shown in FIG. 3(c). A silicone glue (SilPoxy, Smooth On) was used to hold them in place. The part was then placed into the second mold 312 whereupon liquid silicone was poured into the channel, simultaneously submerging the diodes. After curing, it is removed and the top of the channel is covered in gold leaf 314, fully encapsulating the channel with a reflective layer. The final step involved was simply placing the part into the last mold 316 and pouring the liquid silicone to cover the gold leaf 314 at the top of the channel.

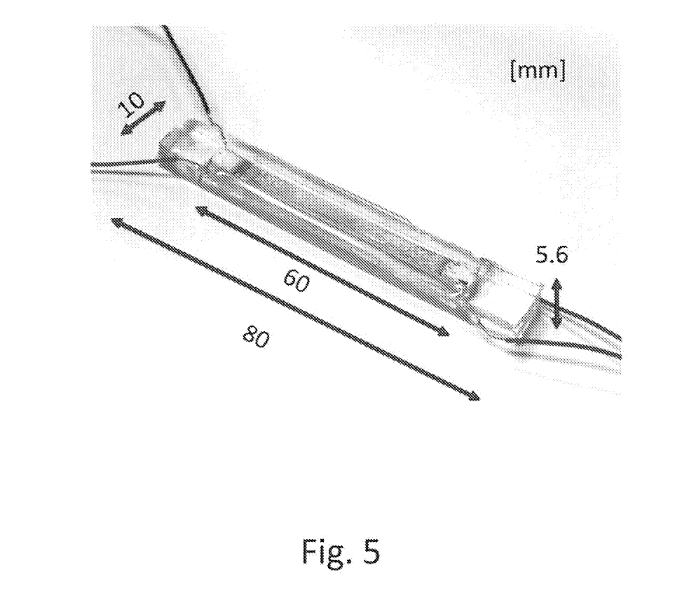

The final product for this embodiment is shown in FIG. 5, having final dimensions being approximately 80 mm in length, 10 mm in width and 5.6 mm in height, and the square channel has a side length of 3 mm each. The actual length of the channel is 60 mm, which is the gage length for testing. The extra 10 mm on each end were used to grasp onto for tensile testing.

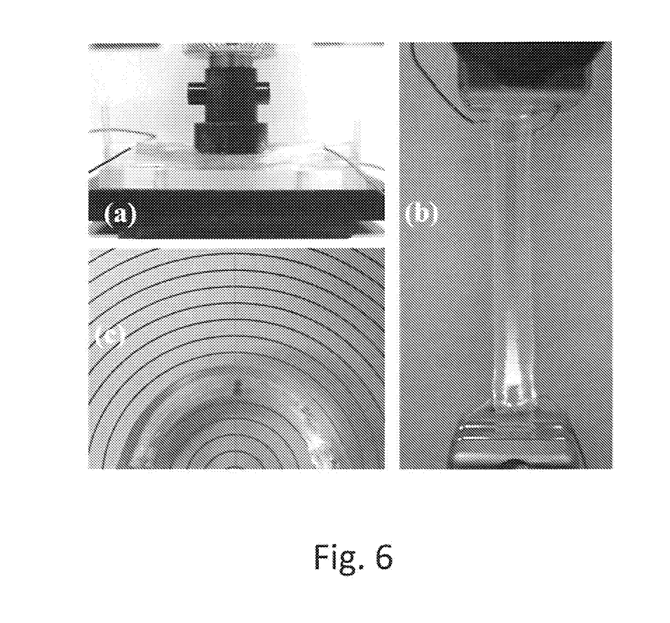

This embodiment was tested for three modes of deformation, pressure, strain, and bending, as shown in FIG. 6. The output signal for each mode was a voltage, which was converted from the photocurrent generated by the photodiode.

FIG. 7 shows a second embodiment of the invention and a simplified method of fabrication. In FIG. 7(a) substrate 700 is molded. The material used is a 9:1 mix of EcoFlex gel and PDMS, to create a highly stretchable and optically transparent silicon hybrid. A black pigment was added to the portions of the device comprising the body (i.e., the material surrounding the clear waveguide) to prevent interference from ambient light entering the waveguide through the microcracks as the device is deformed.

There is a three step process for creating the device as shown in FIG. 7. First, two separate molds were used to fabricate the semi-circular wave guide 702 and substrate layer 700 of the sensor. The substrate layer is shown in FIG. 7(a) and the optical waveguide is shown in FIG. 7(b). Before curing waveguide 702, LED 706 and photodiode 708 were embedded in opposite ends of waveguide 702, as shown in FIGS. 7(b) and 8(a). After curing, waveguide 702 was coated in a thin layer of gold leaf 704, as shown in FIG. 7(b). No extra bonding material was required as the gold leaf 704 adhered well to the naturally tacky substrate 700. Next, the complete waveguide 702 was placed on the top surface of substrate 700 as shown in FIG. 7(b). The device was then placed in another mold where body 710, with a black pigment added, was used to cover waveguide 702, LED 706, photodiode 708 and the exposed portions of substrate 700, to complete the sensor. The completed device is shown in FIGS. 7(c) and 7(d). For each stage of the curing, the molds were placed in a vacuum chamber for several minutes to remove imperfections and air bubbles in the polymer and then cured at room temperature for approximately two hours.

A prototype of the device is shown in FIG. 8 and is approximately 84 mm long, 7 mm wide, and 5 mm tall. The semi-circular wave guide 702 has a diameter of approximately 4 mm and the length of approximately 60 mm. The extra 10 mm on each end of waveguide 702 is useful for clamping for tensile testing and an extra 4 mm to accommodate the LED 706 and photodiode 708. As previously stated, waveguide 702 may have any cross sectional area. However, the preferred shape is driven by the ease of manufacturing. As one of skill in the art would realize, the device's measurements may vary, depending on the intended application.

FIG. 8(a) shows several waveguides having LEDs embedded therein. These waveguides may be used for separate devices, or, in yet another embodiment of the invention, multiple waveguides may be used in a single device to provide the ability to do mixed mode detection, that is, the ability to detect deformations due to combinations of compression, stretching and bending in one device.

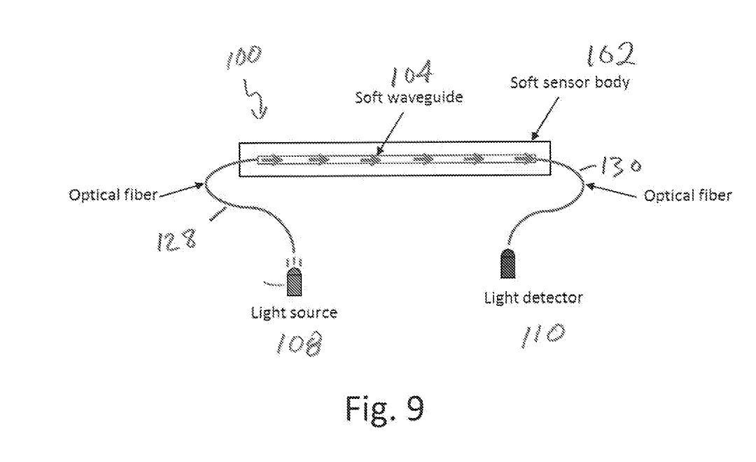

In yet another embodiment of the invention, shown in FIG. 9, the optical components of the invention (i.e., light source 108 and light detector 110) may be located external from the actual device and may be connected to waveguide via optical fiber 108, 110. This allows a reduction in the thickness of the device, as the device no longer needs to accommodate the relatively thick optical components.

In all embodiments of the invention, and representing yet another set of embodiments, the reflective material coating the optical waveguide may be sputtered on instead of being manually applied in leaf form to the outside surface of the waveguide. Thus, the soft waveguides can be made using two different manufacturing techniques: gold leafing and gold sputtering. While gold leafing is simple and cost effective, it involves a manual coating process that may cause some failures and uncontrolled quality. However, gold sputtering provides much more uniform and thin gold layer on a cylindrical waveguide in a much more controlled way. One negative is that the sputtering process is relatively expensive compared with gold leafing.

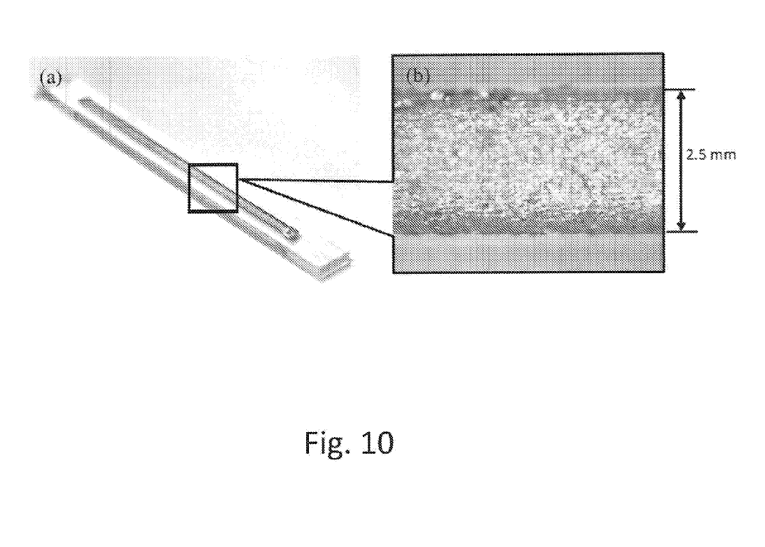

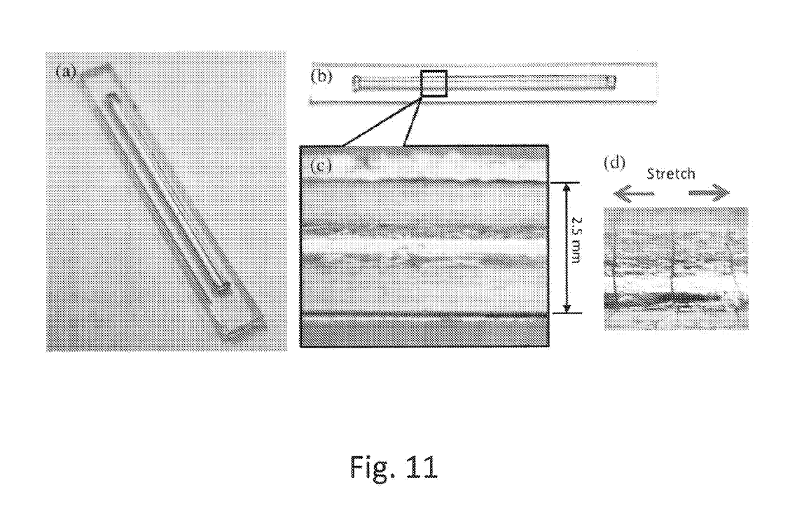

The base material can be soft rubber materials, such as silicone, Polydimethylsiloxane (PDMS), and polyurethane. FIG. 10(a) shows half of a cylindrical soft waveguide made of optically transparent silicone rubber using the gold leafing method. The magnified view in FIG. 10(b) shows many wrinkles in the gold leaf as the result of the leafing process. FIG. 11(c) shows a magnified view of the same sized soft waveguide made of PDMS and by gold sputtering. The thickness of gold leaf before being laminated is 120 nm, but the actual thickness is expected to be thicker due to the wrinkle. The thickness of the sputtered gold layer is 200 nm, however it is much more uniform than the layer show in FIG. 10(b). FIG. 11(d) shows the microcracks in the sputtered gold layer as the device is stretched.

In repeated tests, the signal responses for pressure (compression), strain (stretching) and curvature (bending) show linearity, repeatability, and independence with strain rate. These results, plus the simplicity in fabrication, make the invention a novel replacement for microfluidic sensors.

Several examples of polymer compounds have been specified herein, however, the invention is not intended to be limited to these polymers, or, in fact, to polymers in general. Different applications may require that the body and waveguide of the device vary in elasticity. Additionally, different materials may be used, for example, polyethylene may be a suitable substitute for the silicone-based polymer compounds specified herein.

Although the invention is illustrated and described herein with reference to specific embodiments, the invention is not intended to be limiting to the details shown. Rather, various modifications may be made in the details without departing from the invention.

1. A sensing device for sensing physical deformations, comprising:
a light source;
a photodetector;
an optical waveguide for transmitting light emitted by said light source to said photodetector; and
a body for containing said light source, said photodetector and said optical waveguide.

2. The sensing device of claim 1 wherein said optical waveguide comprises:
a length of a polymer compound; and
a reflective coating composed of an optically reflective material on the outside walls of said length of polymer compound.

3. The sensing device of claim 2 further wherein said body is composed of a polymer compound.

4-8. (canceled)

9. The sensing device of claim 1 wherein said optical waveguide is transparent to light emitted by said light source and further wherein said body is opaque to light emitted by said light source.

10. The sensing device of claim 1 wherein said device is capable of detecting compressive, tensile and bending deformations.

11. The device of claim 1 further comprising multiple optical waveguides including multiple light sources and photodetectors.

12. The sensing device of claim 11 wherein said device is capable of detecting mixed mode deformations.

13. The sensing device of claim 4 wherein said polymer compound is elastic, such that said sensing device returns to its original shape after a force causing said physical deformation is removed.

14. A sensing device for sensing physical deformations comprising:
an optical waveguide wherein said waveguide is generally tubular in shape and is composed of a transparent elastic polymer;
a layer of reflective material disposed on the outside surface of said optical waveguide, said reflective material comprising gold;
a light source embedded in one end of said optical waveguide;
a light detector embedded in the opposite end of said optical waveguide; and
a body surrounding said optical waveguide, said body being composed of said elastic polymer.

15. The sensing device of claim 14 further comprising a pigment, added to said elastic polymer of which said body is composed, said pigment making said body opaque to light.

16. The sensing device of claim 14 wherein said optical waveguide has a semi-circular cross-sectional shape.

17. The sensing device of claim 14 wherein the current through said photodiode is a function of the amount of physical deformation experienced by said device.

18. The sensing device of claim 17 wherein the light received by said photodiode varies as the physical deformation of said device causes microcracks in said reflective coating, thereby allowing a certain amount of light to escape said waveguide, said amount of escaping light being a function of the amount of physical deformation experienced by said device.

19. The sensing device of claim 14 wherein said elastic polymer allows said sensing device to return to its original shape after a force causing said physical deformation is removed.

20. A method of fabricating a sensing device for sensing physical deformations, comprising the steps of:
forming an optical waveguide from a transparent polymer compound;
embedding an LED in one end of said optical waveguide and a photodetector in the opposite end of said optical waveguide;
placing a coating of reflective material on the outside surface of said optical waveguide;
forming a substrate composed of an opaque polymer compound;
placing said optical waveguide on said substrate; and
forming a body composed of said opaque polymer compound such that said body covers said optical waveguide and said substrate.

21. The method of claim 20 wherein said optical waveguide is generally tubular in shape and has a semi-circular cross-sectional shape.

22. The method of claim 20 wherein said reflective coating is composed of gold.

23. (canceled)

24. The method of claim 20 wherein said polymer compound is elastic, such that said sensing device returns to its original shape after a force causing said physical deformation is removed.

25. The method of claim 20 wherein said step of placing a coating of reflective material on the outside surface of said optical waveguide further comprises placing one or more layers of said reflective material in leaf form on the outside surface of said optical waveguide.

26. The method of claim 20 wherein said step of placing a coating of reflective material on the outside surface of said optical waveguide further comprises sputtering said reflective material onto the outside surface of said optical waveguide.

27-28. (canceled)
